SEQUENCE LISTING (10 sequences)

SEQ ID NO: 1 - DNA, 2982 bp, Homo sapiens
cgcaccccgcgcagcggctgagccgggagccagcgcagcctcggccccgcagctcaagcctcgtccccgccgccgccgccgccgccgccgccgccgcccccggggcatggcctgtctgatggccgctttctcggtcggcaccgccatgaatgccagcagttactctgcagagatgacggagcccaagtcggtgtgtgtctcggtggatgaggtggtgtccagcaacatggaggccactgagacggacctgctgaatggacatctgaaaaaagtagataataacctcacggaagcccagcgcttctcctccttgcctcggagggcagctgtgaacattgaattcagggacctttcctattcggttcctgaaggaccctggtggaggaagaaaggatacaagaccctcctgaaaggaatttccgggaagttcaatagtggtgagttggtggccattatgggtccttccggggccgggaagtccacgctgatgaacatcctggctggatacagggagacgggcatgaagggggccgtcctcatcaacggcctgccccgggacctgcgctgcttccggaaggtgtcctgctacatcatgcaggatgacatgctgctgccgcatctcactgtgcaggaggccatgatggtgtcggcacatctgaagcttcaggagaaggatgaaggcagaagggaaatggtcaaggagatactgacagcgctgggcttgctgtcttgcgccaacacgcggaccgggagcctgtcaggtggtcagcgcaagcgcctggccatcgcgctggagctggtgaacaaccctccagtcatgttcttcgatgagcccaccagcggcctggacagcgcctcctgcttccaggtggtctcgctgatgaaagggctcgctcaagggggtcgctccatcatttgcaccatccaccagcccagcgccaaactcttcgagctgttcgaccagctttacgtcctgagtcaaggacaatgtgtgtaccggggaaaagtctgcaatcttgtgccatatttgagggatttgggtctgaactgcccaacctaccacaacccagcagattttgtcatggaggttgcatccggcgagtacggtgatcagaacagtcggctggtgagagcggttcgggagggcatgtgtgactcagaccacaagagagacctcgggggtgatgccgaggtgaacccttttctttggcaccggccctctgaagaggactcctcgtccatggaaggctgccacagcttctctgccagctgcctcacgcagttctgcatcctcttcaagaggaccttcctcagcatcatgagggactcggtcctgacacacctgcgcatcacctcgcacattgggatcggcctcctcattggcctgctgtacttggggatcgggaacgaagccaagaaggtcttgagcaactccggcttcctcttcttctccatgctgttcctcatgttcgcggccctcatgcctactgttctgacatttcccctggagatgggagtctttcttcgggaacacctgaactactggtacagcctgaaggcctactacctggccaagaccatggcagacgtgccctttcagatcatgttcccagtggcctactgcagcatcgtgtactggatgacgtcgcagccgtccgacgccgtgcgctttgtgctgtttgccgcgctgggcaccatgacctccctggtggcacagtccctgggcctgctgatcggagccgcctccacgtccctgcaggtggccactttcgtgggcccagtgacagccatcccggtgctcctgttctcggggttcttcgtcagcttcgacaccatccccacgtacctacagtggatgtcctacatctcctatgtcaggtatgggttcgaaggggtcatcctctccatctatggcttagaccgggaagatctgcactgtgacatcgacgagacgtgccacttccagaagtcggaggccatcctgcgggagctggacgtggaaaatgccaagctgtacctggacttcatcgtactcgggattttcttcatctccctccgcctcattgcctattttgtcctcaggtacaaaatccgggcagagaggtaaaacacctgaatgccaggaaacaggaagattagacactgtggccgagggcacgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggtttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgatggctgcacagtcagatgtctggtggcagagagtccgagcatggagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaaccgagtcacccagctggtctcatacatagacagcacttgtgaaggattgaatgcaggttccaggtggagggaagacgtggacaccatctccactgagccatgcagacatttttaaaagctatacaaaaaattgtgagaagacattggccaactctttcaaagtctttctttttccacgtgcttcttattttaagcgaaatatattgtttgtttcttcctaaaaaaaaaaaaaaaaaaaaaaaaa

SEQ ID NO: 2 - DNA, 3874 bp, Homo sapiens
tggtaggagagcgcagtcgccccgcgggctcggaggacacagcgtccgggaccaggctctgggaagcgggtgtgcacagcccagcagagtcagggcgcgaccccaaccgcctctcaccatccttgccatctcctccgagccgagggccggccctcctgcctcgcctgccccgccccggccccgccccggcccgggccgccgggaccgggagccgggacgcgggtgccgcagcccgaggcaggtaaggggagccccggctctccgtccccggtccgtggcagcgctgagcatcccggccccgcaccggcccgggggcgcgtcaaggtcggcggcgtgatggcggagaaggcgctggaggccgtgggctgtggactagggccgggggctgtggccatggccgtgacgctggaggacggggcggaaccccctgtgctgaccacgcacctgaagaaggtggagaaccacatcactgaagcccagcgcttctcccacctacccaagcgctcagccgtggacatcgagttcgtggagctgtcctattccgtgcgggaggggccctgctggcgcaaaaggggttataagacccttctcaagtgcctctcaggtaaattctgccgccgggagctgattggcatcatgggcccctcaggggctggcaagtctacattcatgaacatcttggcaggatacagggagtctggaatgaaggggcagatcctggttaatggaaggccacgggagctgaggaccttccgcaagatgtcctgctacatcatgcaagatgacatgctgctgccgcacctcacggtgttggaagccatgatggtctctgctaacctgaagctgagtgagaagcaggaggtgaagaaggagctggtgacagagatcctgacggcactgggcctgatgtcgtgctcccacacgaggacagccctgctctctggcgggcagaggaagcgtctggccatcgccctggagctggtcaacaacccgcctgtcatgttctttgatgagcccaccagtggtctggatagcgcctcttgtttccaagtggtgtccctcatgaagtccctggcacaggggggccgtaccatcatctgcaccatccaccagcccagtgccaagctctttgagatgtttgacaagctctacatcctgagccagggtcagtgcatcttcaaaggcgtggtcaccaacctgatcccctatctaaagggactcggcttgcattgccccacctaccacaacccggctgacttcatcatcgaggtggcctctggcgagtatggagacctgaaccccatgttgttcagggctgtgcagaatgggctgtgcgctatggctgagaagaagagcagccctgagaagaacgaggtccctgccccatgccctccttgtcctccggaagtggatcccattgaaagccacacctttgccaccagcaccctcacacagttctgcatcctcttcaagaggaccttcctgtccatcctcagggacacggtcctgacccacctacggttcatgtcccacgtggttattggcgtgctcatcggcctcctctacctgcatattggcgacgatgccagcaaggtcttcaacaacaccggctgcctcttcttctccatgctgttcctcatgttcgccgccctcatgccaactgtgctcaccttccccttagagatggcggtcttcatgagggagcacctcaactactggtacagcctcaaagcgtattacctggccaagaccatggctgacgtgccctttcaggtggtgtgtccggtggtctactgcagcattgtgtactggatgacgggccagcccgctgagaccagccgcttcctgctcttctcagccctggccaccgccaccgccttggtggcccaatctttggggctgctgatcggagctgcttccaactccctacaggtggccacttttgtgggcccagttaccgccatccctgtcctcttgttctccggcttctttgtcagcttcaagaccatccccacttacctgcaatggagctcctatctctcctatgtcaggtatggctttgagggtgtgatcctgacgatctatggcatggagcgaggagacctgacatgtttagaggaacgctgcccgttccgggagccacagagcatcctccgagcgctggatgtggaggatgccaagctctacatggacttcctggtcttgggcatcttcttcctagccctgcggctgctggcctaccttgtgctgcgttaccgggtcaagtcagagagatagaggcttgccccagcctgtaccccagcccctgcagcaggaagcccccagtcccagccctttgggactgttttaaccttatagacttgggcactggttcctggcggggctatcctctcctcccttggctcctccacaggctggctgtcggactgcgctcccagcctgggctctgggagtgggggctccagccctccccactatgcccaggagtcttcccaagttgatgcggtttgtagcttcctccctactctctccaacacctgcatgcaaagactactgggaggctgctgcctccttcctgcccatggcaccctcctctgctgtctgcctgggagccctaggctctctagggccccacttacaactgaccaaagtggccccctctgggggtccccaccacacaagtgtttgtaaactgggctgctataaggttggagttccagggctgggccctggtggagtccactggaagtcccattatggatgttgaaatggacagggaaggactctggaagtctcttcctcctcctcctcttctctccacccctagaccctggctgacttggacaatctgccaggacagaagctgggttttctgtctaggtcaccactcccaatcctggggattggagaggcctggggctgtgggatgccccatccccctccccatcacctttggtgggggcagggcctggtggcacctgtgcaataatgtctgtgtttctctcccacctgccactggaactggagaatgcactttattctgggcggggggtgagtgggggaagacccaaccctcctttctcgctgcccctaacgcatgcacggtctcgtgatgctccctccctctccggagtgacaggcacatacatgagaacaggccatctcagccctacacacttgccatcccctacagcacagaggaagagtgatggtggcatgctggtggtggcgggtgctggtgggaggacagtgccaacctcctcctggggatcccatgttggagactctaaggataaggctggtgctgcccagggtgtctacaggaactgcaggtgtctacccccaagtcttccctcctcccaagccaggggtggcacagggcactagatccctggagttcaggaaccaacacaagcacaaccacgggcataagttggccttggccactgccacccacggccctccttttgtgctccatgctggcatcttcactcccctaccccttccccagccactgctgctcattcaaacttctgtccatgtccctccactgttcctatcagcaggtggcccctgggcatcagaacagcctgccctgggcaccaggtggcagacacactcagagcatgtctggctttcctggtgggtccaggctcattctgcttctgatttcccctcccccagggctcattttccccctttttcctgtacacatccctgtctacctcctctcaccctgccacagattcttcctatcacacagggatgccagttgtatttgtgggatttcacccattattaataaaacctatatttatacagtatgcaatgtgtaaaaaaaaaaaaaaaaaaaaaaaaaaaaaaaaaaaaaaaa

SEQ ID NO: 3 - DNA, 3586 bp, mouse
gaattcctcgttcctgcaacctcgtccccgccgcccgcagacttgtctccactgccgctgccgccgccgcccccggggcatggcctgtctgatggccgctttctcggtcggcaccgccatgaatgccagcagctactctgccgcaatgacggagcccaagtccgtgtgcgtgtcagtggacgaggtcgtgtccagcaacgtggatgaggttgagacagacctgctcaatgggcacctgaagaaggtggacaacaacttcacagaggcccagcgcttttcctcccttccgcggagggcggccgtgaacatcgaattcaaggacctttcctactctgtacccgaggggccctggtggaagaagaaaggatacaagacccttttgaaagggatctctgggaaattcaacagtggagagctggtggccatcatgggtccttctggagctgggaagtccacactcatgaatattctggcaggatacagggagactggcatgaaaggggcagtccttatcaatggaatgccccgggacctgcgctgcttccggaaggtctcctgctacatcatgcaggacgacatgctgctgcctcacctcactgttcaggaggccatgatggtgtccgcgcatctgaagctgcaggagaaggatgaaggcagacgggagatggtcaaagagatcctgacagccctgggcttgctgccctgtgccaacacacgcacggggagcctctcaggcggccagcggaaacgcctggccattgccctggagctggtcaacaacccgcctgtcatgttctttgatgagcccaccagtggcctggacagcgcctcctgcttccaagtggtgtctctgatgaaaggactggcccagggtggccgctccatcgtctgtaccatccaccagcccagtgccaagctctttgagctctttgaccagctttatgtcctaagtcaaggacaatgcgtatacaggggaaaggtctccaatctcgtgccgtatctgagggatctgggtctgaactgccctacctaccacaacccagcagactttgtcatggaagtggcatcaggggagtacggggatcagaacagtcgcctggtgagagccgtgcgagagggcatgtgtgacgctgactataagagagacctcgggggcgacaccgatgtgaacccgtttctttggcaccggcctgctgaagaggactccgcctccatggaaggttgccatagcttctcggccagctgcctcacccagttctgcatcctcttcaagaggaccttcctcagcatcatgcgggactcggtcctgacacatctgcgaatcacctcgcacattgggatcggcctgctcattggcctgctgtacctggggattgggaatgaagccaagaaggtccttagcaactccggcttcctgttcttctccatgctgttcctcatgtttgctgccctcatgcccactgttctgacctttcccctggagatgagtgtcttcctccgggagcacctgaactactggtacagcctgaaggcctactacctggcaaagaccatggccgatgtcccctttcagatcatgttccctgtggcctactgcagtatcgtatactggatgacgtcccagccgtcggacgctgtgcgttttgtgctgttcgctgctctgggtaccatgacatcgctggtggcccagtccttaggactactgattggagctgcatccacatccctgcaggttgcgacatttgtgggtcccgtgacagccatccccgtcctgctcttctccggattctttgtcagctttgacaccatcccagcctacctgcagtggatgtcctacatctcctatgtcagatacggctttgagggggtcatcctgtccatctacggcttggaccgagaagacctgcactgcgacatcgcggagacatgccacttccagaagtcagaggccatcctgagggagctggacgtggagaatgcgaagctgtacctggatttcatcgtcctgggcatcttcttcatctccctgcggctcatcgcctatttcgtcctcagatacaaaatccgggctgagaggtaaaaaccgcctccaagccagcaacgaggcaaagcagacattgtgaccaagggcactgctgggaggtagcagccgacctgctcctggtgacacagactctcccaacccaacctggaggccacgcaagtctgacgatgaccagtgttgagcgcctctgcccgccgggttgaaactgttcgtttccttttctaactaggaagatgtaggacggttgggtttttttttttctttttttttttttttttagatgtgggattcaaaatacaactggcatagaatgccatcctgtgctccagctggggacagtggcttcctcccggagtccaggacacactggccccggatgaggagtgttagccgagttggtccccagagaggtggatatcttggggagaaggtccacacctagcgggcacagaggccacctgaggaccatcttctcttaaggtctcagccttctaaagttcctctcatttctgatgaagtcactttccgagccaaagtcgataaatttcattcacttcgagagattgtatctttttagtacttcttgagggacttattcagggtaaaagatgtgttttgcttagaaacagaagagtccaaccgagtcaccaaggaggcctcacaaataaaaccagagaaggtcaaatgcaggctccgggtgagggagacaccacaccagctgcgctggggcagcagggactaacgcaacgcaatgcaacgcaatgcagacagtgctggggtacttggcaggctcctcccagacttcctttctcatgtgcttcttagcttaaaactcttcctagttttccagccactttcattttgcccaagaaatactcgatccaccttgtgatgtttaaagacccatttcccctttcttcatgaatcacattaactacctccaccgagcctgggaagccggcctggggacatatcgggtgtatccctccaacttgaggggagccgtggggtgtcaaggatgggcaggccggctgagtggaaggggaggtgccatggatgtccccgttaagaaaatgcagctgctcgcctcttccagagcaccaccatctaaggtcagcctcctacaaaagtctctatttttgagggtaggtcagtttaacccctgtgagcctcagtttccccacctgtaagtaattgcactagatcatgtcttgacaggtcctgagattcctcggctgtggccttgatgctttcgtatcttatctgtagagaatgataagggtccgaattttacttaccgcatggataatgtgctttggaactctttagagattctttttattttttacactcctagatcttttttatagagaacacacaattgaaatgcatattatgatgtaattttcggtttgcttgttctatacaaacacggccttgccttctagactcagcttttaaccagggacacgattcgcctttataaaaaaaaaaagtcttcattaaaaaagaaaacaccaaaaaaaaaaaaaaaaaaaaaaa

SEQ ID NO: 4 - DNA, 3628 bp, mouse
gatcctttctgacccgagccctgccttcctctactgcccgccctggcctagccccgccccggcccgggccaccgggacagggagccggggcgcgggtgccgcagccagaggcaggtgaagagagccggagtgtccacctcccgcgccgcaacgctgagcatccgggtcgaacatcccggttgagcatctctgctgagcatccttgcgtcgcagcggccggagacgcgtcaaggccggctgcgtgatggcggagaaggcattggaggccgtgggctgtggactgggccctggggctgtggccatggctgtgacactggaggacggggcagagccccctgtgctgaccacgcacctaaagaaggtggagaaccatatcacagaagcacagcgcttctctcacctgcccaagcgctctgctgtggacatcgagttcgtggaactgtcctattccgtgcgggaggggccctgctggcgcaaacggggttacaagaccctcctcaagtgcctgtctggtaagttctgtcgccgagagctgattggcatcatgggcccctctggggctggcaagtccacattcatgaacatcttggcagggtacagggagtcggggatgaaggggcagatcctggttaacgggaggccgcgcgagctgaggaccttccgcaagatgtcctgctacatcatgcaggatgacatgctactgcctcacctcacagtcctggaggccatgatggtctctgccaacctgaagctgagtgagaagcaagaggtgaagaaggaactggtgacagagatcctgacagccctgggactgatgtcctgctctcacacgaggacagctctgctctctggagggcagaggaagcgcctggccattgctctggaactggtcaacaacccgcctgtcatgttctttgatgagcctaccagcggtctagacagcgcttcttgtttccaagtggtgtccctcatgaagtccctggcccatgggggccgcaccgtcatctgcaccatccaccagcccagtgccaagctctttgagatgtttgacaagctctacatcctgagccaggggcaatgcatcttcaagggcgtggttaccaacctgattccctatctgaaggggcttggcttgcactgtcccacctaccacaaccccgctgacttcatcattgaggtggcctctggagagtatggagacctgaaccccatgctgttcagggctgtgcagaatggcctttgcaccatggccgagaagaagagcagccctgggaagaatgagctccctgcccactgccccacttgccctccggagctggatcctattgaaagccacacatttgccaccagcaccctaacccagttctgcatcctcttcaggaggactttcttgtccatcctcagggacacggtcctgacccacttgcggttcatgtcacatgtgttgatcggcgtcctcattggcctcctctacctgcacattggcgatgatgccagcaaagtcttcaacaacaccggcttcctcttcttttccatgctcttcctcatgtttgcagccctcatgccgaccgtgctcacctttcccttagagatggcggtcttcatgagggagcacttgaactactggtatactctcaaagcctattacttggccaagaccatggctgatgtgcccttccaggtggtatgcccagtggtgtactgcagcatcgtgtactggatgacaggccagccagccgagaccagccgcttcctgctcttctcagccttggccatagccaccgccttagttgcccagtctttgggactcttgattggcgctgcctctacctccttgcaggtggccacttttgtgggcccagtgactgccatccctgtcctcttgttctccggcttcttcgtcagtttcaggaccatccccacttacttgcaatggagctcctacctctcctatgttaggtatggctttgagggtctgatcctgaccatctatggtatggagcgaggacacctgacctgcttagatgaacagtgcccattccgggacccacaaatcattttgcgtgagctggatgtagaggaggccaaactctacatggacttcctggtcctgggcattttcttccttgccctgcggctgctggcataccttgtgctccggtaccgggtcaagtctgagagatagagcctggctcctacctcttctgggcaggaaggccccagttccagccctttggggctgttttaaacttgtacttgggcattggttgctggtgcagctgccctcctctccctcggctgctactcaggctggctggctgtgggctgtgtctcccagtctcagccccggaggtagggctccagccctcccgtcaaggccaggagtcttcccaagttgatgcagttggtagcttcttccctactgtctccaacacctgcatgcaaagactgctgggaggttgctgccctcctctgcggaaagcaccctcctctgttgtttgcctgggagctccacgttctctaggccttgacttacaagtgatcagattgactcccctctggggtccccgccacactctgctatcaggttggagtcccagggctgggccctggtggggtctccagggagtcccatcatggacattgaatggagcaaggagggactctggaagcttcctcctctttctctttccctcccaacacccactttggctaatttggactatctgttgggacagaagccgggctttctgtctgggccaccactcccaatcctggagattggagagagcataggatgtcccggccccttccctaagccttcggtctggatagacctcaacatcacctctgcaataatgtctgtgtcactctcccatcggccacaggacctggagaatgcactttattctggggggtgaggcggggaagcccccatcctccttccttgctgctcctggggctcagttccacaggctcctttcctctccagaatcacatcacaggcacacaaaggccgccaggcaatctcagccctacacaggcaccatcccttgtggctcagaggaagagtggccgtggcagggagcagtcccaacgtcctcctgcagaatcccactttggaggctaagaatgagactggtgtgacccagtatgactccaggctcggcaggtgtctatctcccagtcttccctccctcttgagcccggggtggcgctggccttctggaggtcagaacccaacaagcacaagaatgggcatagctggcctcggccactgctcccccatggccctccttttgtgccccaagctgacactcactcacctgcccctcctccagctgcctgctgctcactcaaacttctgttcacatgtattcatcttccggtcagcagctggttcctggacatcagagcggcggggcctggacatcaggtggcagacacacctgtctatcttcttggtgggtccaggatcatcctatatctggtttctcctcctccaggactctttccccagtccctgtgctcactcctctgtatctgccccaggttcttctcattacacagagaccagttgtatttgtgggatttcacccattattaataaaacctatatttatacagc

SEQ ID NO: 5 - protein, 19 aa, mouse
CGTGGATGAGGTTGAGACA

SEQ ID NO: 6 - protein, 19 aa, mouse
GGTGGACAACAACTTCACA

SEQ ID NO: 7 - protein, 19 aa, mouse
GAAGGTGGAGAACCATATC

SEQ ID NO: 8 - protein, 19 aa, mouse
GCACTTGAACTACTGGTAT

SEQ ID NO: 9 - DNA, 21 bp, mouse
tcgtatcttatctgtagagaa

SEQ ID NO: 10 - DNA, 26 bp, mouse
ccgggtcaagtcaagtctgagagata

(19) United States
(12) Patent Application Publication
(10) Pub. No.: US 2007/0094743 A1
(43) Pub. Date: Apr. 26, 2007

(54) ABCG1/ABCG4-related methods and compositions

(76) Inventors: Nan Wang, Flushing, NY (US); Alan R. Tall, Cresskill, NJ (US)

Correspondence Address: COOPER & DUNHAM, LLP, 1185 AVENUE OF THE AMERICAS, NEW YORK, NY, 10036, US

(21) Appl. No.: 11/471,170

(22) Filed: Jun. 19, 2006

Related U.S. Application Data
(60) Provisional application No. 60/692,438, filed on Jun. 20, 2005.

(51) Int. Cl. A01K 67/027; C12N 5/06

(52) U.S. Cl. 800/14; 800/18; 435/353; 435/354

(57) ABSTRACT

This invention provides a non-macrophage cell having therein an expression vector encoding ABCG1, wherein the non-macrophage cell expresses ABCG1. This invention further provides a non-macrophage cell having therein an expression vector encoding ABCG4, wherein the non-macrophage cell expresses ABCG4. This invention further provides related transgenic animals and methods.

This application claims the benefit of U.S. Provisional Application No. 60/692,438, filed Jun. 20, 2005, the contents of which are incorporated herein by reference into the subject application.

The invention disclosed herein was made with government support under National Institutes of Health Grant No. HL 54591. Accordingly, the U.S. Government has certain rights in this invention.

Throughout this application, various publications are referenced by Arabic number. Full citations for these publications may be found at the end of the specification immediately preceding the claims. The disclosures of these publications in their entireties are hereby incorporated by reference in order to more fully describe the state of the art.

### BACKGROUND OF THE INVENTION

A major theory to account for the inverse relationship between high-density lipoprotein (“HDL”) levels and cardiovascular risk is that HDL promotes the efflux of cholesterol from arterial wall macrophage foam cells and decreases atherosclerosis. This hypothesis appeared to be supported by the discovery that Tangier Disease, a disorder characterized by very low HDL levels, macrophage foam cell accumulation and increased atherosclerosis, is caused by mutations in the ATP-binding cassette transporter (“ABCA1”) (1-4). ABCA1 mediates efflux of cellular phospholipids and cholesterol to lipid-poor apolipoproteins, such as apoA-I and apoE (5, 6), initiating the formation of HDL. However, ABCA1 interacts poorly with HDL-2 and HDL-3 particles (5, 7) that constitute the bulk of the plasma HDL, and ABCA1 variants are not likely to account for a major part of the genetic variation in HDL levels in the general population (8). Thus, the activity of ABCA1 does not readily account for cholesterol efflux from foam cells to HDL and the mechanism underlying the inverse relationship between HDL levels and atherosclerosis risk remains uncertain.

The oxysterol-activated transcription factors liver X receptor/retinoid X receptor (“LXR/RXR”) induce expression of ABCA1, as well as a number of other molecules involved in cellular cholesterol efflux, transport and excretion (9, 10). Treatment of macrophages with LXR activators increased net cholesterol efflux to HDL-2, suggesting the presence of unique LXR target genes mediating cholesterol efflux to HDL (11). Some ABCG family members are also LXR/RXR targets, such as ABCG5 and ABCG8, the defective genes in sitosterolemia (12-14). ABCG family members are half-transporters, largely of unknown function. Particularly, it was not known whether different members of the ABCG transporter family might be responsible for cellular cholesterol efflux to HDL.

### SUMMARY OF THE INVENTION

This invention provides a non-macrophage cell having therein an expression vector encoding ABCG1, wherein the non-macrophage cell expresses ABCG1.

This invention further provides a non-macrophage cell having therein an expression vector encoding ABCG4, wherein the non-macrophage cell expresses ABCG4.

This invention further provides a transgenic, non-human mammal wherein the somatic cells which would normally express ABCG1 do not express ABCG1.

This invention further provides a transgenic, non-human mammal whose ABCG1-expressing somatic cells, upon introduction of a suitable inducing agent to the mammal, cease expressing ABCG1.

This invention further provides a transgenic, non-human mammal having a tissue comprising ABCG1-expressing somatic cells wherein, upon introduction of a suitable inducing agent to the tissue, the cells of the tissue cease expressing ABCG1.

This invention further provides a transgenic, non-human mammal having a tissue comprising somatic cells which do not express ABCG1, wherein the non-ABCG1-expressing cells of the tissue comprise somatic cells which, in a non-transgenic mammal, would express ABCG1.

This invention further provides a transgenic, non-human mammal wherein the somatic cells which would normally express ABCG4 do not express ABCG4.

This invention further provides a transgenic, non-human mammal whose ABCG4-expressing somatic cells, upon introduction of a suitable inducing agent to the mammal, cease expressing ABCG4.

This invention further provides a transgenic, non-human mammal having a tissue comprising ABCG4-expressing somatic cells wherein, upon introduction of a suitable inducing agent to the tissue, the cells of the tissue cease expressing ABCG4.

This invention further provides a transgenic, non-human mammal having a tissue comprising somatic cells which do not express ABCG4, wherein the non-ABCG4-expressing cells of the tissue comprise somatic cells which, in a non-transgenic mammal, would express ABCG4.

This invention further provides a method for producing an ABCG1-expressing, non-macrophage cell which comprises introducing into a non-macrophage cell an expression vector encoding ABCG1.

This invention further provides a method for producing an ABCG4-expressing, non-macrophage cell which comprises introducing into a non-macrophage cell an expression vector encoding ABCG4.

This invention further provides a method for treating a subject afflicted with Alzheimer's disease comprising administering to the subject a therapeutically effective amount of an agent that increases the amount of ABCG1 activity in the subject's cells, thereby treating Alzheimer's disease.

This invention further provides a method for treating a subject afflicted with Alzheimer's disease comprising administering to the subject a therapeutically effective amount of an agent that increases the amount of ABCG4 activity in the subject's cells, thereby treating Alzheimer's disease.

This invention further provides a method for determining whether an agent increases ABCG1 activity comprising (a) contacting the agent, in the presence of HDL, with a cholesterol-loaded, non-macrophage cell having therein an expression vector encoding ABCG1, wherein the non-macrophage cell expresses ABCG1, under conditions permitting ABCG1-mediated cholesterol efflux from the cell, (b) measuring the amount of cholesterol efflux from the cell resulting from step (a) and (c) comparing the amount of cholesterol efflux measured in step (b) with the amount of cholesterol efflux resulting in the absence of the agent, wherein a higher amount of cholesterol efflux in the presence of the agent indicates that the agent increases ABCG1 activity.

Finally, this invention provides a method for determining whether an agent increases ABCG4 activity comprising (a) contacting the agent, in the presence of HDL, with a cholesterol-loaded, non-macrophage cell having therein an expression vector encoding ABCG4, wherein the non-macrophage cell expresses ABCG4, under conditions permitting ABCG4-mediated cholesterol efflux from the cell, (b) measuring the amount of cholesterol efflux from the cell resulting from step (a), and (c) comparing the amount of cholesterol efflux measured in step (b) with the amount of cholesterol efflux resulting in the absence of the agent, wherein a higher amount of cholesterol efflux in the presence of the agent indicates that the agent increases ABCG4 activity.

### BRIEF DESCRIPTION OF THE FIGURES

FIGS. 1A-1B LXR/RXR activation increases macrophage cholesterol efflux to HDL independent of ABCA1. (A, B) Cholesterol efflux to apoA-I (15 μg/ml) (A) or HDL (25 μg protein/ml) (B) was determined in mouse peritoneal macrophages isolated from wild type or ABCA1^{-/-} mice. The cells were labeled with [³H]cholesterol in cell culture media plus 10% fetal bovine serum for 16 hours, then treated with or without 5 μM T0901317 plus 5 μM 9-cis retinoic acid for 16 hours followed by cholesterol efflux for 4 hours.

FIGS. 2A-2D Cells transfected with ABCG1 and ABCG4 cDNAs show increased cholesterol efflux to HDL. HEK293 cells were transiently transfected with plasmid constructs expressing ABCG transporters or control empty vector (mock) and cholesterol efflux was initiated by addition of HDL to media. (A), [³H]cholesterol efflux to HDL-2 or HDL-3 (25 μg/ml HDL protein) or media alone (control) for 4 hours. (B), [³H]cholesterol efflux to HDL-2 (25 μg/ml HDL protein) for 4 hours. (C), [³H]cholesterol efflux to HDL-2 at indicated concentrations for 4 h. (D), [³H]cholesterol efflux to HDL-2 (25 μg/ml HDL protein) for the indicated period of time.

FIGS. 3A-3B ABCG1 and ABCG4 expression increase cholesterol mass efflux to HDL and decrease cellular cholesterol content. (A), Free cholesterol mass in culture media determined in transfected 293 cells incubated with HDL-2 (25 μg/ml HDL protein) for 4 hours. (B), Total cellular cholesterol was determined after 6 hour incubation of transfected 293 cells with HDL-2 (25 μg/ml).

FIGS. 4A-4D ABCG1 and ABCG4 do not promote cholesterol efflux to apoA-I and do not bind HDL while promoting cholesterol efflux to HDL, LDL and cyclodextrin. (A), HDL-2 (25 μg/ml HDL protein) and apoA-I (15 μg/ml protein) mediated cholesterol efflux during a 4 h incubation with 293 cells expressing ABCG transporters. (B), [³H]choline-containing phospholipid efflux to HDL-2 (25 μg/ml HDL protein) during a 4 h incubation with 293 cells expressing ABCG transporters. (C), Cholesterol efflux to HDL (25 μg/ml HDL protein), LDL (25 μg/ml LDL protein) or cyclodextrin (1 mM) during a 4 h incubation with 293 cells expressing ABCG transporters. (D), [¹²⁵I]-HDL binding to 293 cells expressing SR-BI or ABCG transporters.

FIGS. 5A-5B Suppression of ABCG1 expression by RNAi decrees macrophage cholesterol efflux to HDL. [³H]cholesterol efflux to HDL-2 was determined using mouse peritoneal macrophages after transfection of the cells with synthetic siRNA against ABCG1 (A, B). Two different concentrations of siRNA were used, as indicated. As a control, scrambled siRNA or siRNA against ABCA7 were used. mRNA levels of ABCG1 (B) normalized against β-actin mRNA from macrophages treated with 120 nM siRNA were determined by Taqman real-time RT-PCR. *, P<0.05; **, P<0.01.

FIG. 6 Human ABCG1 nucleotide sequence

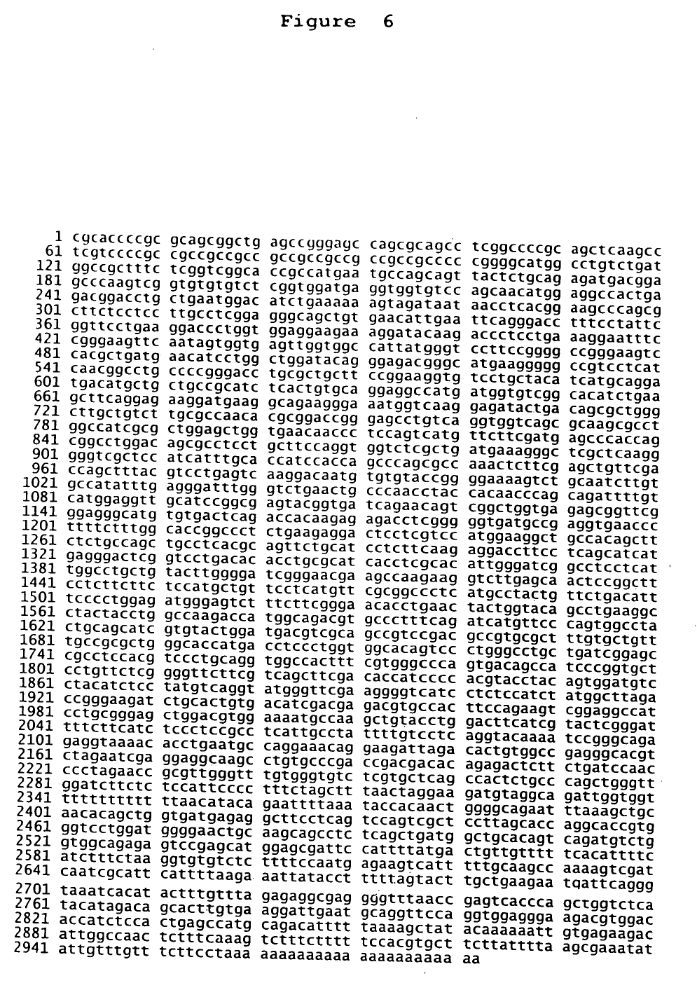

FIG. 7 Human ABCG4 nucleotide sequence

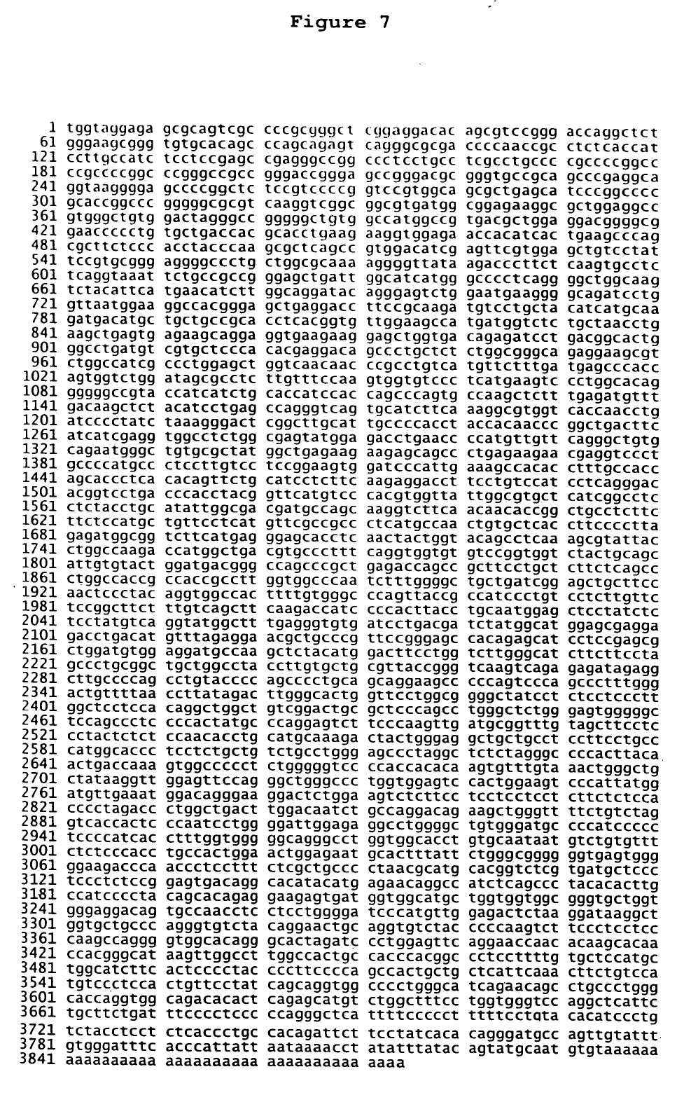

FIG. 8 Mouse ABCG1 nucleotide sequence

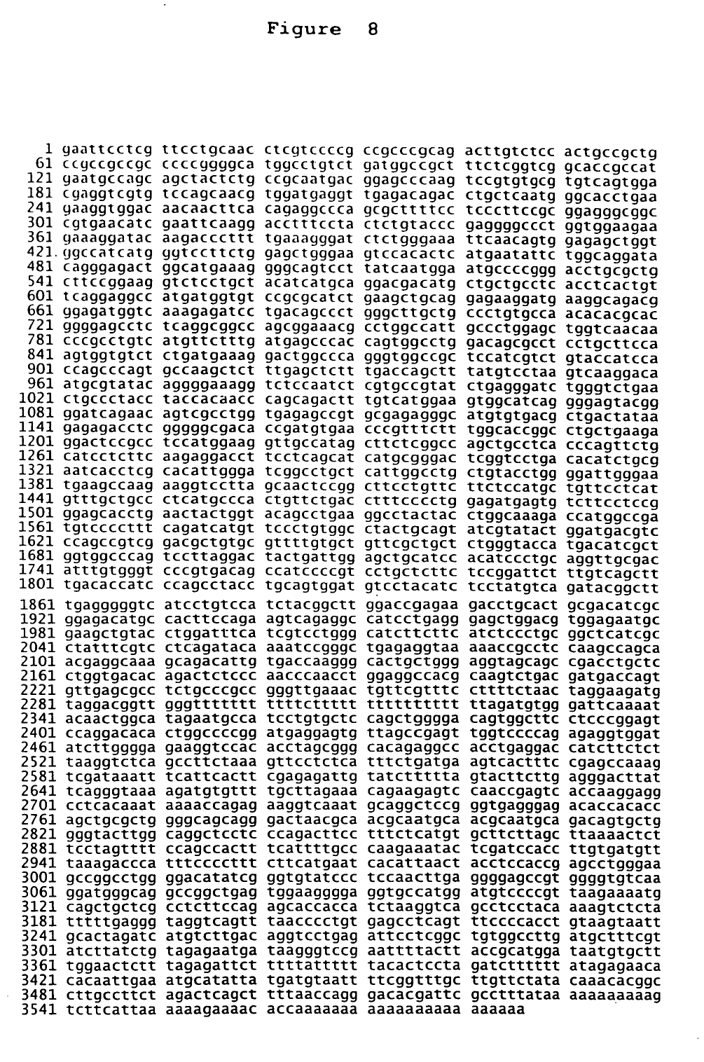

FIG. 9 Mouse ABCG4 nucleotide sequence

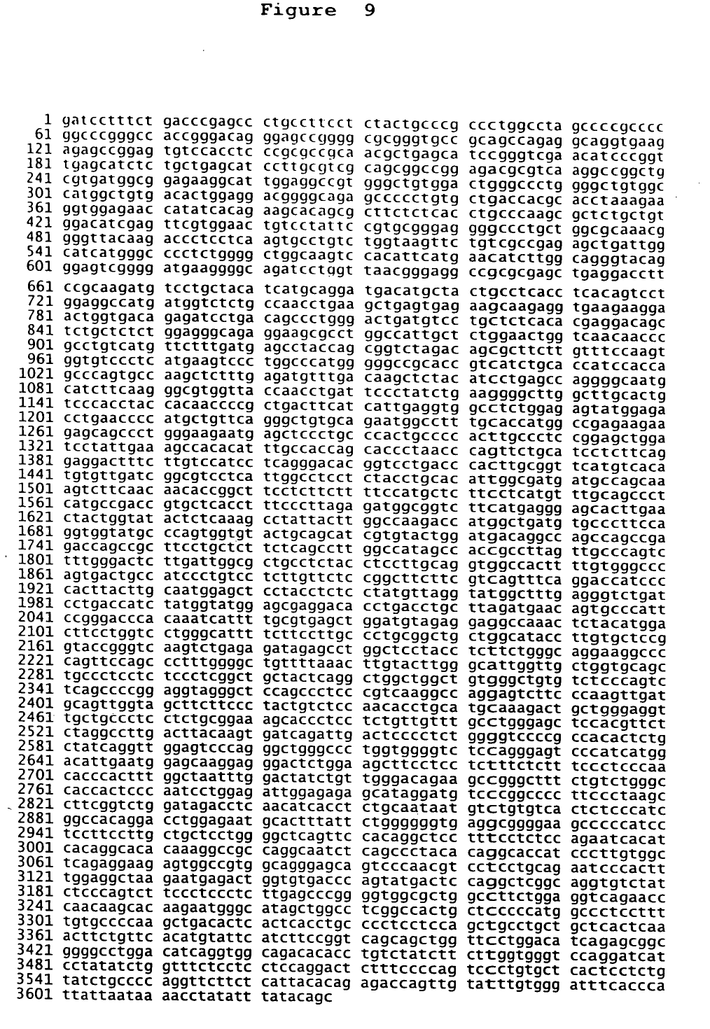

### DETAILED DESCRIPTION OF THE INVENTION

Terms

“ABCG1” is used herein to mean “ATP-binding cassette transporter G1”.

“ABCG1 activity” shall include, without limitation, any catalytic activity performed by ABCG1. One example of ABCG1 activity is the facilitation of cholesterol or phospholipid efflux from a cell.

“ABCG4” is used herein to mean “ATP-binding cassette transporter G4”.

“ABCG4 activity” shall include, without limitation, any catalytic function performed by ABCG4. One example of ABCG4 activity is the facilitation of cholesterol or phospholipid efflux from a cell.

“Administering” an agent can be effected or performed using any of the various methods and delivery systems known to those skilled in the art. The administering can be performed, for example, intravenously, orally, nasally, via the cerebrospinal fluid, via implant, transmucosally, transdermally, intramuscularly, and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“Agent” shall mean any chemical entity, including, without limitation, a glycomer, a protein, an antibody, a lectin, a nucleic acid, a small molecule, and any combination thereof. Examples of possible agents include, but are not limited to, a ribozyme, a DNAzyme and an siRNA molecule.

“Bacterial cell” shall mean any bacterial cell. One example of a bacterial cell is E. coli.

“Cholesterol efflux-mediating protein” shall mean any protein which, when properly situated in and/or on a cell, facilitates the efflux of cholesterol from the cell (i.e., the movement of cholesterol from the cell to the cell's exterior). Examples of a “cholesterol efflux-mediating protein” include, without limitation, ABC1, ABCG1 and ABCG4.

“Expression vector” shall mean a nucleic acid encoding a nucleic acid of interest and/or a protein of interest, which nucleic acid, when placed in a cell, permits the expression of the nucleic acid or protein of interest. For example, a bacterial expression vector includes a promoter such as the lac promoter and for transcription initiation the Shine-Dalgarno sequence and the start codon AUG. Similarly, a eukaryotic expression vector includes a heterologous or homologous promoter for RNA polymerase II, a downstream polyadenylation signal, the start codon AUG and a termination codon for detachment of the ribosome. Such vectors may be obtained commercially or assembled from the sequences described in methods well-known in the art.

A cell expressing “little” of a specific protein, i.e., ABCG1 or ABCG4, includes, for example, a cell expressing (i) a trace amount of such protein or (ii) an amount of such protein insufficient to ensure the cell's survival via cholesterol efflux were the cell to become cholesterol-loaded.

“Macrophage-like cell” includes, for example, a cell which shares some, but not all, morphological and functional characteristics with a macrophage cell. Macrophage-like cells can be derived from mouse or human tumor cell lines.

“Mammalian cell” shall mean any mammalian cell. Mammalian cells include, without limitation, cells which are normal, abnormal and transformed, and are exemplified by neurons, epithelial cells, muscle cells, blood cells, immune cells, stem cells, osteocytes, endothelial cells and blast cells.

“Non-macrophage cell” shall mean any cell other than a macrophage cell. Examples of non-macrophage cells are hepatocytes and adipocytes.

“Nucleic acid” shall mean any nucleic acid molecule, including, without limitation, DNA (e.g., cDNA), RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Polypeptide” and “protein” are used interchangeably herein, and each means a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation.

“Subject” shall mean any organism including, without limitation, a mammal such as a mouse, a rat, a dog, a guinea pig, a ferret, a rabbit and a primate. In the preferred embodiment, the subject is a human being.

“Therapeutically effective amount” means an amount sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. In one embodiment, the therapeutically effective amount is from about 1 mg of agent/subject to about 1 g of agent/subject per dosing. In another embodiment, the therapeutically effective amount is from about 10 mg of agent/subject to 500 mg of agent/subject. In a further embodiment, the therapeutically effective amount is from about 50 mg of agent/subject to 200 mg of agent/subject. In a further embodiment, the therapeutically effective amount is about 100 mg of agent/subject. In still a further embodiment, the therapeutically effective amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art.

“Treating” a disorder means slowing, stopping or reversing the progression of the disorder, and/or ameliorating symptoms associated with a disorder.

### Embodiments of the Invention

This invention provides a non-macrophage cell having therein an expression vector encoding ABCG1, wherein the non-macrophage cell expresses ABCG1. In one embodiment, the ABCG1 is human ABCG1. In another embodiment, the cell is a bacterial cell, a yeast cell, an insect cell or a mammalian cell. In another embodiment, the cell is a macrophage-like cell. In another embodiment, the non-macrophage cell does not express ABCG1 prior to having the expression vector introduced into it. In another embodiment, the non-macrophage cell expresses little ABCG1 prior to having the expression vector introduced into it. In another embodiment, the non-macrophage cell does not express any cholesterol efflux-mediating protein prior to having the expression vector introduced into it.

This invention further provides a non-macrophage cell having therein an expression vector encoding ABCG4, wherein the non-macrophage cell expresses ABCG4. In one embodiment, the ABCG4 is human ABCG4. In another embodiment, the cell is a bacterial cell, a yeast cell, an insect cell or a mammalian cell. In another embodiment, the cell is a macrophage-like cell.. In another embodiment, the non-macrophage cell does not express ABCG4 prior to having the expression vector introduced into it. In another embodiment, the non-macrophage cell expresses little ABCG4 prior to having the expression vector introduced into it. In another embodiment, the non-macrophage cell does not express any other cholesterol efflux-mediating protein prior to having the expression vector introduced into it.

This invention further provides a transgenic, non-human mammal wherein the somatic cells which would normally express ABCG1 do not express ABCG1. In one embodiment, the mammal is a mouse or a rat.

This invention further provides a transgenic, non-human mammal whose ABCG1-expressing somatic cells, upon introduction of a suitable inducing agent to the mammal, cease expressing ABCG1. In one embodiment, the mammal is a mouse or a rat.

This invention further provides a transgenic, non-human mammal having a tissue comprising ABCG1-expressing somatic cells wherein, upon introduction of a suitable inducing agent to the tissue, the cells of the tissue cease expressing ABCG1. In one embodiment, the mammal is a mouse or a rat. In another embodiment, the tissue is liver tissue. In another embodiment, the tissue is brain tissue.

This invention further provides a transgenic, non-human mammal having a tissue comprising somatic cells which do not express ABCG1, wherein the non-ABCG1-expressing cells of the tissue comprise somatic cells which, in a non-transgenic mammal, would express ABCG1. In one embodiment, the mammal is a mouse or a rat. In another embodiment, the tissue is liver tissue. In another embodiment, the tissue is brain tissue.

This invention further provides a transgenic, non-human mammal wherein the somatic cells which would normally express ABCG4 do not express ABCG4. In one embodiment, the mammal is a mouse or a rat.

This invention further provides a transgenic, non-human mammal whose ABCG4-expressing somatic cells, upon introduction of a suitable inducing agent to the mammal, cease expressing ABCG4. In one embodiment, the mammal is a mouse or a rat.

This invention further provides a transgenic, non-human mammal having a tissue comprising ABCG4-expressing somatic cells wherein, upon introduction of a suitable inducing agent to the tissue, the cells of the tissue cease expressing ABCG4. In one embodiment, the mammal is a mouse or a rat. In another embodiment, the tissue is liver tissue. In another embodiment, the tissue is brain tissue.

This invention further provides a transgenic, non-human mammal having a tissue comprising somatic cells which do not express ABCG4, wherein the non-ABCG4-expressing cells of the tissue comprise somatic cells which, in a non-transgenic mammal, would express ABCG4. In one embodiment, the mammal is a mouse or a rat. In another embodiment, the tissue is liver tissue. In another embodiment, the tissue is brain tissue.

This invention further provides a method for producing an ABCG1-expressing, non-macrophage cell which comprises introducing into a non-macrophage cell an expression vector encoding ABCG1. In one embodiment, the ABCG1 is human ABCG1. In another embodiment, the cell is a bacterial cell, a yeast cell, an insect cell or a mammalian cell. In another embodiment, the cell is a macrophage-like cell. In another embodiment, the non-macrophage cell does not express ABCG1 prior to having the expression vector introduced into it. In another embodiment, the non-macrophage cell expresses little ABCG1 prior to having the expression vector introduced into it. In another embodiment, the non-macrophage cell does not express any cholesterol efflux-mediating protein prior to introducing the expression vector into the non-macrophage cell.

This invention further provides a method for producing an ABCG4-expressing, non-macrophage cell which comprises introducing into a non-macrophage cell an expression vector encoding ABCG4. In one embodiment, the ABCG4 is human ABCG4. In another embodiment, the cell is a bacterial cell, a yeast cell, an insect cell or a mammalian cell. In another embodiment, the cell is a macrophage-like cell. In another embodiment, the non-macrophage cell does not express ABCG4 prior to having the expression vector introduced into it. In another embodiment, the non-macrophage cell expresses little ABCG4 prior to having the expression vector introduced into it. In another embodiment, the non-macrophage cell does not express any cholesterol efflux-mediating protein prior to introducing the expression vector into the non-macrophage cell.

This invention further provides a method for treating a subject afflicted with Alzheimer's disease comprising administering to the subject a therapeutically effective amount of an agent that increases the amount of ABCG1 activity in the subject's cells, thereby treating Alzheimer's disease. In one embodiment, the agent is a PPAR agonist. In another embodiment, the agent is an LXR activator. In another embodiment, the subject is human. In another embodiment, the cells are neuronal cells.

This invention further provides a method for treating a subject afflicted with Alzheimer's disease comprising administering to the subject a therapeutically effective amount of an agent that increases the amount of ABCG4 activity in the subject's cells, thereby treating Alzheimer's disease. In one embodiment, the agent is a PPAR agonist. PPAR agonists include, without limitation, pioglitazone and rosiglitazone. In another embodiment, the agent is an LXR activator. An example of an LXL activator is TO901317. In another embodiment, the subject is human. In another embodiment, the cells are neuronal cells.

This invention further provides a method for determining whether an agent increases ABCG1 activity comprising (a) contacting the agent, in the presence of HDL, with a cholesterol-loaded, non-macrophage cell having therein an expression vector encoding ABCG1, wherein the non-macrophage cell expresses ABCG1, under conditions permitting ABCG1-mediated cholesterol efflux from the cell, (b) measuring the amount of cholesterol efflux from the cell resulting from step (a) and (c) comparing the amount of cholesterol efflux measured in step (b) with the amount of cholesterol efflux resulting in the absence of the agent, wherein a higher amount of cholesterol efflux in the presence of the agent indicates that the agent increases ABCG1 activity. In one embodiment, the ABCG1 is human ABCG1. In another embodiment, the HDL is HDL-2 or HDL-3. In another embodiment, the cell does not express any other cholesterol efflux-mediating protein. In another embodiment, the cell is a bacterial cell, a yeast cell, an insect cell or a mammalian cell.

Finally, this invention provides a method for determining whether an agent increases ABCG4 activity comprising (a) contacting the agent, in the presence of HDL, with a cholesterol-loaded, non-macrophage cell having therein an expression vector encoding ABCG4, wherein the non-macrophage cell expresses ABCG4, under conditions permitting ABCG4-mediated cholesterol efflux from the cell, (b) measuring the amount of cholesterol efflux from the cell resulting from step (a) and (c) comparing the amount of cholesterol efflux measured in step (b) with the amount of cholesterol efflux resulting in the absence of the agent, wherein a higher amount of cholesterol efflux in the presence of the agent indicates that the agent increases ABCG4 activity. In one embodiment, the ABCG4 is human ABCG4. In another embodiment, the HDL is HDL-2 or HDL-3. In another embodiment, the cell does not express any other cholesterol efflux-mediating protein. In another embodiment, the cell is a bacterial cell, a yeast cell, an insect cell or a mammalian cell.

This invention will be better understood from the Experimental Details which follow. However, one skilled in the art will readily appreciate that the specific methods and results discussed are merely illustrative of the invention as described more fully in the claims which follow thereafter.

Experimental Details

Synopsis

The mechanisms responsible for the inverse relationship between plasma HDL levels and atherosclerotic cardiovascular disease are poorly understood. ABCA1 mediates efflux of cellular cholesterol to lipid-poor apolipoproteins, but not to HDL particles that constitute the bulk of plasma HDL. This study shows that two ABC transporters of unknown function, ABCG1 and ABCG4, mediate isotopic and net mass efflux of cellular cholesterol to HDL. In transfected 293 cells, ABCG1 and ABCG4 stimulate cholesterol efflux to both smaller (HDL-3) and larger (HDL-2) subclasses, but not to lipid-poor apoA-I. Treatment of macrophages with an LXR activator results in up-regulation of ABCG1 and increases cholesterol efflux to HDL. RNA interference reduced expression of ABCG1 in LXR-activated macrophages and caused a parallel decrease in cholesterol efflux to HDL. These studies indicate that ABCG1 and ABCG4 promote cholesterol efflux from cells to HDL. ABCG1 is highly expressed in macrophages and probably mediates cholesterol efflux from macrophage foam cells to the major HDL fractions, providing a mechanism to explain the relationship between HDL levels and atherosclerosis risk.

Material and Methods

Plasma Lipoprotein Preparations

HDL-2 (density 1.063-1.125 g/ml) and HDL-3 (density 1.125-1.210 g/ml) were isolated by preparative ultracentrifugation from normolipidemic human plasma and stored in phosphate buffered saline containing 1 mM EDTA. Low-density lipoprotein (“LDL”) was from Biomedical Technologies Inc. (Stoughton, Mass.). ABCA1^{-/-} mice were provided by Dr. Omar Francone (Pfizer, Groton, Conn.) and macrophages isolated from the wild type and knockout littermates were used for the experiments.

Plasmid Constructs and Cell Transfection

The plasmid constructs expressing mouse ABCG transporters were prepared by cloning mouse full-length cDNAs into pCMV-sport6 vector, and the cDNA sequence was confirmed by DNA sequencing. For transient transfection of human embryonic kidney (“HEK”) 293 cells, cells in 12- or 24-well collagen-coated plates were transfected with various plasmid constructs with LipofectAMINE 2000 (InVitrogen, Calif.) at 37° C. overnight (˜20 h). To estimate transfection efficiency, a construct expressing green fluorescent protein (“GFP”) was routinely used in the experiment to visually monitor for transfection efficiency. The transfection efficiency of HEK293 cells was in the range of 60-80% of cells. Although transfection efficiency did vary from experiment to experiment, variations within the same experiment were small.

Cellular Lipid Efflux Assays

Generally, HEK293 cells were labeled by culturing for 24 h in 10% fetal bovine serum/Dulbecco's modified Eagle's medium media containing either 2 μCi/ml [³H]cholesterol for cholesterol efflux or 2 μCi/ml [³H]choline (1Ci=37 GBq) for phospholipid efflux. The next day, cells were washed with fresh media and then HDL, LDL or cyclodextrin were added as acceptor and incubated for the indicated period before the media and cells were collected for analysis. Phospholipid and cholesterol efflux were expressed as the percentage of the radioactivity released from the cells into the medium relative to the total radioactivity in cells plus medium. For cholesterol mass efflux, the collected media were extracted with hexane:isopropanol (3:2, vol/vol) with β-sitosterol (5 μg/sample) added as the internal standard. The recovered lipid fractions were dried under nitrogen gas, 100 μl of chloroform was added, and the samples were subject to gas-liquid chromatographic analysis. For HDL cell association, cells were incubated with [¹²⁵I]HDL (1.5 pg/ml) in 0.2% bovine serum albumin/DMEM media for 1 h at 37° C. After washing three times with fresh media, cells were lysed with 0.1% SDS and 0.1 M NaOH lysis buffer, and radioactivity was determined by γ-counter. To determine the free cholesterol mass in media after cholesterol efflux in the presence or absence of HDL, the lipid fraction was extracted from the media with hexane:isopropanol (3:2). After drying under nitrogen gas, the mass of free cholesterol dissolved in chloroform was determined using gas chromatography.

Small Interfering RNA (si)RNA-Mediated Macrophage RNA Interference (RNAi)

cRNA oligonucleotides derived from the mouse ABCG1 and ABCG4 target sequences were obtained from Dharmacon (Lafayette, Colo.) and used to induce RNAi to suppress ABCG1 and ABCG4 expression in thioglycollate-elicited mouse peritoneal macrophages. Two target sequences were selected using the program from Dharmacon: 5′CGTGGATGAGGTTGAGACA3′ and 5′GGTGGACAACAACTTCACA3′ for ABCG1; and 5′GAAGGTGGAGAACCATATC3′ and 5′GCACTTGAACTACTGGTAT3′ for ABCG4.

Where indicated, RNA oligonucleotides targeting both sequences were mixed and used to down-regulate ABCG1 or ABCG4 gene expression. The scrambled control RNA oligonucleotides also were obtained from Dharmacon. An independent set of siRNA targeting ABCG1 (5′TCGTATCTTATCTGTAGAGAA3′) or ABCG4 (5′CCGGGTCAAGTCAAGTCTGAGAGATA3′) was obtained from Qiagen and used where indicated. For cholesterol efflux assays, mouse peritoneal macrophages were plated in 24- or 48-well plates and cultured in 10% fetal bovine serum and DMEM media at 37° C. for 24 hours. Cells were then transfected with siRNA and LipofectAMINE 2000 at the indicated concentration and labeled with isotopic cholesterol (2 μCi/ml [³H]cholesterol in 1% fetal bovine serum) in the presence or absence of TO901317 (2 μM) for 48 hours. Cells were washed twice and equilibrated for 30 minutes for the third wash, and then HDL or other acceptors were added for the indicated period. Levels of ABCG1 and ABCG4 mRNAs normalized against β-actin mRNA were determined using Taqman real-time quantititative RT-PCR. The primers and probes were from Applied Biosystems.

Results

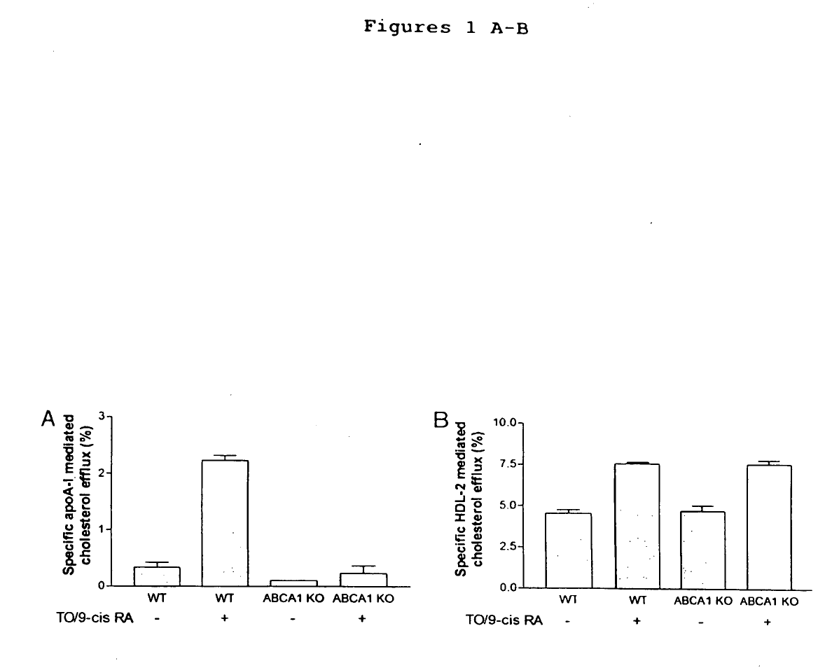

It was previously shown that LXR activation in macrophages resulted in increased cholesterol efflux to HDL-2 (11). Because HDL-2 does not appear to interact with ABCA1 in transfected 293 cells (7), it was possible that LXR activation might induce an alternative pathway, leading to increased cholesterol efflux to HDL. To more directly test this possibility, macrophages from wild type or ABCA1^{-/-} mice were treated with LXR/RXR activators, and then cholesterol efflux to apolipoprotein (apo)A-I or HDL-2 was determined. LXR/RXR activation induced cholesterol efflux to both apoA-I (FIG. 1A) and to HDL-2 (FIG. 1B). Whereas deficiency of ABCA1 virtually abolished cholesterol efflux to apoA-I, there was no effect on cholesterol efflux to HDL, confirming an ABCA1-independent, LXR-induced efflux pathway to HDL. Previous studies ruled out a role of apoE or SR-BI in this process since LXR/RXR activation led to increased cholesterol efflux to HDL-2 in apoE^{-/-} macrophages or macrophages treated with scavenger receptor (SR)-BI-neutralizing antibody. This finding led to the evaluation of the possibility that unique ABC transporters that are LXR targets might mediate cholesterol efflux to HDL. Members of the ABCG family have been implicated in cholesterol transport (12), and several members of this family are known LXR targets (13, 15).

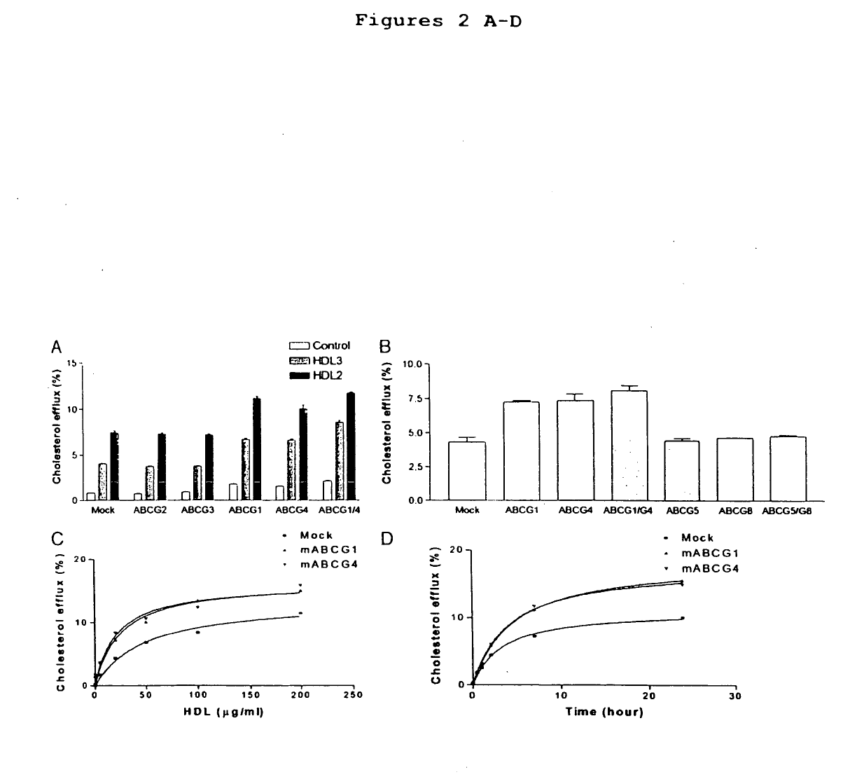

In order to examine the hypothesis that an ABCG transporter family member might be responsible for cholesterol efflux to HDL, all six members of the family that are expressed in mammalian cells were cloned, and each cDNA was transiently expressed in HEK293 cells labeled with isotopic cholesterol. Incubation of mock-transfected 293 cells with plasma HDL caused efflux of isotopic cholesterol (FIG. 2A), likely reflecting passive exchange of cholesterol between HDL and cells. Transient transfection with ABCG1 or ABCG4 resulted in stimulation of isotopic cholesterol efflux to both HDL-2 and HDL-3 (FIG. 2A). HDL-specific cholesterol efflux (total minus control) was approximately doubled for HDL-3, whereas efflux to HDL-2 was increased ≈50% (FIG. 2B). The combination of ABCG1 and ABCG4 resulted in a further small increase in cholesterol efflux. In contrast, other ABCG transporters that were examined, ABCG2, ABCG3 (FIG. 2A), ABCG5, ABCG8 or ABCG5/ABCG8 co-expression (FIG. 2B), did not promote cholesterol efflux to HDL. Combination of ABCG1 or ABCG4 with any of the other ABCG transporters did not lead to a further increase in cholesterol efflux to HDL (data not shown). Time- and concentration-dependence experiments showed that efflux mediated by ABCG1 or ABCG4 continued to increase over 24 hours, and reached a maximum at ≈50 μg/ml HDL protein, similar to the concentration of HDL present in interstitial fluid (16) (FIGS. 2C, 2D).

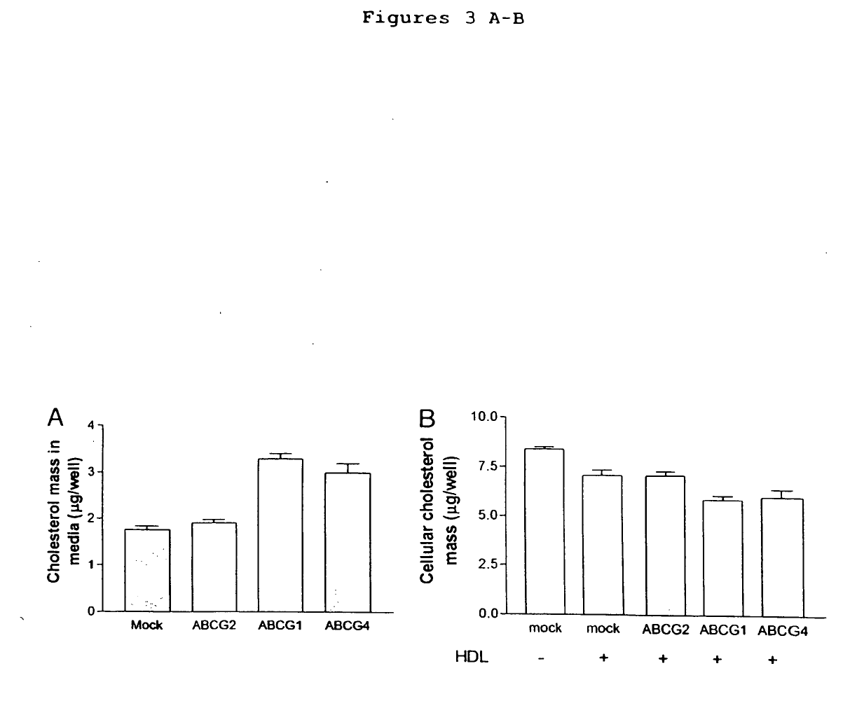

The efflux of isotopic cholesterol can result either from a net transfer process or from exchange of free cholesterol between the cell and HDL. Remarkably, gas-chromatographic measurement of cholesterol content in media indicated almost a doubling of HDL-free cholesterol mass after incubation with cells expressing ABCG1 or ABCG4, indicating a marked stimulation of net free cholesterol efflux (FIG. 3A). A similar increase in free cholesterol mass was observed both for total HDL (FIG. 3A) and for HDL-2 (data not shown). Total cellular cholesterol mass in transfected 293 cells following HDL-mediated cholesterol efflux was also determined (FIG. 3B). HDL treatment slightly decreased cellular cholesterol mass (FIG. 3B) and ABCG1 or ABCG4 expression further reduced the cellular cholesterol content (FIG. 3B), reflecting the increased cholesterol efflux to HDL.

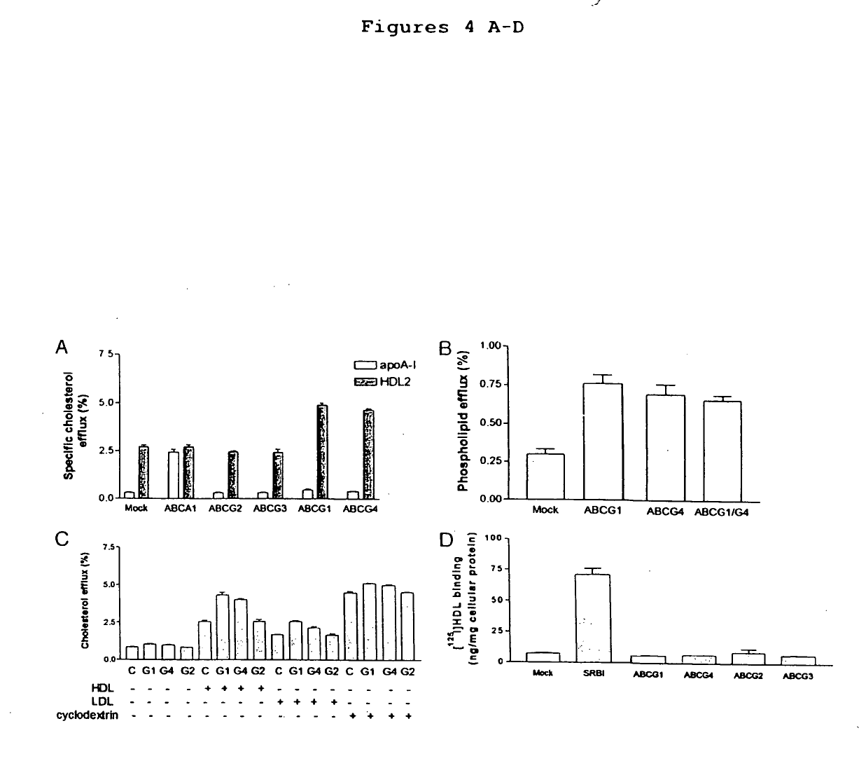

When incubated with lipid-poor apoA-I, cells transfected with ABCG1, ABCG4 or the other ABCG transporters, did not stimulate cholesterol efflux to apoA-I (FIG. 4A). In marked contrast, ABCA1 stimulated efflux to apoA-I but not HDL-2, as reported (7). Cell transfection with ABCG1 or ABCG4 also resulted in a slight increase in efflux of phospholipid radioactivity to HDL (FIG. 4B). However, this transfection represented less than 1% of cellular phospholipid; by comparison, cells transfected with ABCA1 typically efflux several percentage of both cellular phospholipid and cholesterol to apoA-I (5, 7). Thus, ABCG1 and ABCG4 mediate prominent net cholesterol efflux to HDL but not to lipid-poor apoA-I.

The ability of ABCG1 and ABCG4 to stimulate cholesterol efflux to LDL and to an inert cholesterol acceptor, cyclodextrin was also determined (FIG. 4C). In cells transfected with ABCG1 or ABCG4, there was a small but significant stimulation of cholesterol efflux to LDL and to cyclodextrin, but this amount was less than observed with HDL. ABCA1 binds lipid-poor apolipoproteins, and this activity is closely correlated with its ability to mediate lipid efflux from cells (5, 17, 18). Similarly, SR-BI binds HDL, and this appears to be required for its selective uptake function (19). In contrast, cells transfected with ABCG1 or ABCG4 did not bind HDL above control levels (FIG. 4D).

The finding that ABCG1 and ABCG4 promote cholesterol efflux to HDL-2, but not to apoA-I, could explain earlier observations, suggesting an LXR-induced, ABCA1-independent pathway of cholesterol efflux in macrophages (FIG. 1 and (11)). ABCG1 and ABCG4 are expressed in macrophages and are induced by LXR/RXR activation with 22-OH cholesterol and 9-cis-retinoic acid (15, 20, 21). A specific LXR activator, T0901317 (2 μM) increased mRNA levels of ABCG1 and ABCG4 by 3- and 2-fold in mouse macrophages (data not shown), confirming the previous findings.

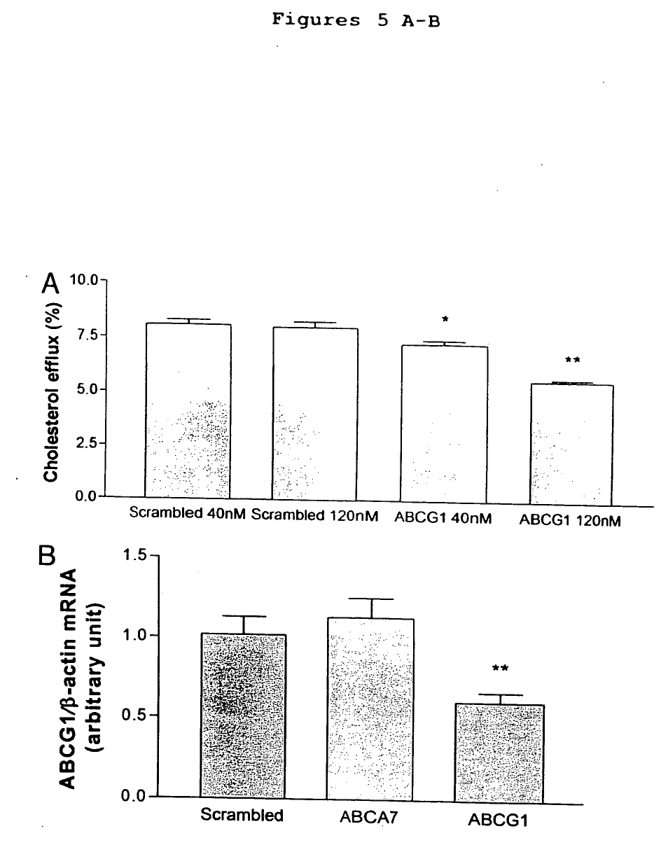

In order to determine if the induction of cholesterol efflux to HDL-2 is due to expression of ABCG1 and/or ABCG4, RNA interference induced by synthetic small interfering RNA (siRNA) in mouse peritoneal macrophages pretreated with the LXR activator TO901317 (2 μM) was used. Knock-down experiments were conducted at two different concentrations of siRNA (40 and 120 nM). Suppression of ABCG1 resulted in a dose-dependent significant reduction in cholesterol efflux to HDL (FIG. 5A). At the higher dose, the suppression resulted in about a 30% reduction in isotopic efflux to HDL. By contrast, RNAi using scrambled RNA or an irrelevant ABCA7 target sequence did not change cholesterol efflux. Measurements of mRNA levels by real-time PCR indicated a specific suppression of ABCG1 mRNA, with ≈50% reduction at the higher level of RNAi (FIG. 5B). Initial results with siRNA against ABCG4 showed a reduced macrophage cholesterol efflux to HDL. However, ABCG1 mRNA was also reduced by the ABCG4 siRNAs (data not shown), probably due to homology in the target sequences (22). An independent set of siRNAs targeting different sequences in ABCG1 and ABCG4 confirmed decreased cholesterol efflux to HDL (data not shown). Because the residual efflux of isotopic cholesterol likely includes a large component due to passive exchange processes, these data suggest that ABCG1, and possibly ABCG4, makes a major contribution to HDL-mediated cholesterol efflux in LXR-induced macrophages.

Discussion

Previous studies suggested the existence of an LXR-induced, ABCA1-independent pathway of cholesterol efflux to HDL (11). This hypothesis was confirmed by comparing efflux in wild type and ABCA1^{-/-} macrophages (FIG. 1). Using cell transfection and RNA interference, it is now shown that two known LXR targets of unknown function, ABCG1 and ABCG4, mediate cholesterol efflux to the major. HDL fractions HDL-2 and HDL-3 but not to lipid-poor apoA-I. In contrast, ABCA1 mediates cholesterol efflux to apoA-I and interacts poorly with HDL-2 and HDL-3 (5, 7). The ability of ABCG1, and possibly ABCG4, to mediate cholesterol efflux to HDL could be important in the athero-protective effect of HDL because the bulk of plasma HDL consists of such mature HDL particles.

While it has been speculated that ABCG1 could have a role in cellular cholesterol efflux and reverse cholesterol transport (23, 24), no definite function of either ABCG1 or ABCG4 has been previously assigned. ABCG1 was initially identified as a macrophage LXR target (15). Schmitz and co-workers reported that an antisense oligodeoxynucleotide to ABCG1 reduced macrophage cholesterol efflux to HDL-3 (21). However, this group subsequently stated that the same oligodeoxynucleotide also reduced expression of apoE and questioned whether ABCG1 was directly involved in lipid efflux (23). Hepatocyte overexpression of ABCG1 by adenovirus infection in mice resulted in a slight lowering of HDL and increased biliary cholesterol secretion (24, 25). Physiological relevance of these experiments is somewhat uncertain because hepatic expression of ABCG1 is probably predominantly in Kupffer cells (26), while adenovirus is expressed mainly in hepatocytes. Thus, the function of ABCG1 has remained enigmatic and its role in reverse cholesterol transport is considered uncertain (24).

The present study suggests a major role of ABCG1 in HDL-mediated cholesterol efflux in macrophages while the role of ABCG4 is less certain. Although ABCG4 mRNA is detectable in this and other studies using RT-PCR methodology (20), these semiquantitative measurements suggest a low level of ABCG4 expression in mouse macrophages even after LXR activation (data not shown). However, ABCG4 is highly expressed in brain (27) and HDL-like particles are present in cerebrospinal fluids (28, 29). Therefore, ABCG4 could promote cholesterol efflux to these HDL particles in brain. This finding is of particular interest in light of recent studies of the role of cholesterol metabolism in development of Alzheimer's disease which suggest that promotion of cholesterol efflux in neuronal cells decreases amyloid β peptide formation and secretion (30).

In addition to ABCG1 and ABCG4, it was shown previously that SR-BI also facilitates cholesterol efflux to HDL, but not to lipid-poor apoA-I (31). SR-BI promotes the bidirectional flux of cholesterol between cells and HDL and when HDL is phospholipid-rich and cholesterol-poor, net cholesterol efflux can result. However, unlike the findings with ABCG1 and ABCG4, SR-BI does not create a gradient of cholesterol concentration from cells to HDL. Studies with bone marrow transplantation show an increased atherosclerotic lesion area in apoE^{-/-}-recipient mice treated with SR-BI^{-/-} apoE^{-/-} donor cells compared with apoE^{-/-}-recipient mice receiving SR-BI^{+/+} apoE^{-/-} donor cells (32). However, the SR-BI knockout macrophages display no difference in cholesterol efflux to HDL compared with wild type macrophages (32), suggesting that macrophage SR-BI does not have a major role in cholesterol efflux to HDL in mice.

ABCG1 is most closely related to ABCG4, and both may be mammalian homologs of the Drosophila white gene. ABCG transporters are thought to function either as heterodimers, e.g. ABCG5/ABCG8 (33), or homodimers/homomultimers, e.g. ABCG2 (34). Because overexpression of either ABCG1 or ABCG4 resulted in cholesterol efflux to HDL, it appears they can function as homodimers. Also, inconsistent with function as heterodimers, the distribution of the two mRNAs in various tissues does not appear to be strongly correlated (27, 35), and they did not make functional partners with other ABCG family members in our cell expression experiments. However, several different transcripts of ABCG1 are present in macrophages (35, 36), raising the possibility of different functions and possibly heterodimerization with other half-transporters. Moreover, different functions in various cell types and tissues are also possible (26).

When comparing the ability of different acceptors to take up cholesterol, it was found that in addition to efflux to HDL, ABCG1 and ABCG4 caused a slight but significant stimulation of cholesterol efflux to LDL and to an inert cholesterol acceptor, cyclodextrin (FIG. 2). Thus, ABCG1 and ABCG4 can promote cholesterol efflux to a variety of lipoprotein and nonlipoprotein acceptors, suggesting that these transporters may increase availability of cholesterol at the plasma membrane, or at sites that are readily accessible to the plasma membrane. Although specific binding of HDL to ABCG1 and ABCG4 was not observed, HDL can bind cell membranes by nonspecific lipid-lipid interactions (37), perhaps acting to facilitate cholesterol efflux in ABCG1- or ABCG4-expressing cells (38). Rapid and slow components of cholesterol efflux to cyclodextrin have been described (39). The slow component of efflux is ATP-dependent and may reflect cholesterol movement from the endocytic recycling compartment to the plasma membrane (40).

At equal protein concentrations, the level of cholesterol efflux to LDL was ≈55% of that observed for HDL (FIG. 3). Since HDL protein concentration in plasma normally exceeds that of LDL, this suggests that HDL will represent the major acceptor in normal plasma. However, in subjects with high LDL and low HDL levels, cholesterol efflux to LDL could predominate, giving rise to a futile cycle if LDL particles are subsequently ingested by macrophages.

Epidemiological studies indicate that both HDL-2 and HDL-3 are inversely related to atherosclerosis risk (41). Much of the difference in HDL levels between individuals reflects different levels of HDL-2, and HDL levels are in major part genetically determined by variation at the hepatic lipase and apoA-I/apoC-III/apoA-IV loci (8). Since ABCA1 does not directly interact with the main fraction of HDL (5), and does not likely account for a major part of the genetic variation in HDL levels in the general population (8, 42), ABCA1-HDL interactions or associations do not readily explain the protective effect of HDL. In contrast, the demonstration that ABCG1 promotes net cellular cholesterol efflux to HDL has the potential to provide a mechanistic understanding of the relationship of HDL to atherosclerosis risk. HDL infusions in humans are being carried out with reconstituted HDL particles (43) and HDL-raising therapies such as niacin (44) or cholesterol ester transfer protein (“CETP”) inhibition (45) primarily block HDL catabolism and cause an increase in larger particles that are unlikely to interact with ABCA1. CETP inhibitors are in advanced human trials (46). These findings suggest a mechanism to explain how these HDL-directed therapies could lead to cholesterol efflux from macrophage foam cells.

### REFERENCES

1. Orso, E., et al., (2000) Nat. Genet. 24, 192-196.

2. Rust, S., et al., (1999) Nat. Genet. 22, 352-355.

3. Bodzioch, M., et al., (1999) Nat. Genet. 22, 347-351.

4. Brooks-Wilson, A., et al., (1999) Nat. Genet. 22, 336-345.

5. Wang, N., et al., (2000) J. Biol. Chem. 275, 33053-33058.

6. Oram, J. F., et al., (2000) J. Biol. Chem. 275, 34508-34511.

7. Wang, N., et al., (2001) J. Biol. Chem. 276, 23742-23747.

8. Cohen, J. C., et al., (1994) J. Clin. Invest. 94, 2377-2384.

9. Costet, P., et al., (2000) J. Biol. Chem. 275, 28240-28245.

10. Repa, J. J., et al., (2000) Science 289, 1524-1529.

11. Chen, W., et al., (2001) J. Biol. Chem. 276, 43564-43569.

12. Berge, K. E., et al., (2000) Science 290, 1771-1775.

13. Repa, J. J., et al., (2002) J. Biol. Chem. 277, 18793-18800.

14. Yu, L., et al., (2003) J. Biol. Chem. 278, 15565-15570.

15. Kennedy, M. A., et al., (2001) J. Biol. Chem. 276, 39438-39447.

16. Reichl, D. (1990) Eur. Heart J. 11, Suppl. E, 230-236.

17. Chroni, A., et al., (2004) Biochemistry 43, 2126-2139.

18. Fitzgerald, M. L., et al., (2004) J. Lipid Res. 45, 287-294.

19. Krieger, M. (2001) J. Clin. Invest. 108, 793-797.

20. Engel, T., et al., (2001) Biochem. Biophys. Res. Commun. 288, 483-488.

21. Klucken, J., et al., (2000) Proc. Natl. Acad. Sci. U.S.A. 97, 817-822.

22. Jackson, A. L., et al., (2003) Nat. Biotechnol. 21, 635-637.

23. Schmitz, G., et al., (2001) J. Lipid Res. 42, 1513-1520.

24. Brewer, H. B., Jr. & Santamarina-Fojo, S. (2003) Am. J. Cardiol. 91, 3E-11E.

25. Ito, T. (2003) Drug News Perspect. 16, 490-492.

26. Hoekstra, M., Kruijt, et al., (2003) J. Biol. Chem. 278, 25448-25453.

27. Oldfield, S., et al., (2002) Biochim. Biophys. Acta 1591, 175-179.

28. Borghini, I., et al., (1995) Biochim. Biophys. Acta 1255, 192-200.

29. Koch, S., et al., (2001) J. Lipid Res. 42, 1143-1151.

30. Sun, Y., et al., (2003) J. Biol. Chem. 278, 27688-27694.

31. Ji, Y., et al., (1997) J. Biol. Chem. 272, 20982-20985.

32. Zhang, W., et al., (2003) Circulation 108, 2258-2263.

33. Graf, G. A., et al., (2003) J. Biol. Chem. 278, 48275-48282.

34. Xu, J., et al., (2004) J. Biol. Chem. 279. 19781-19789.

35. Savary, S., et al., (1996) Mamm. Genome 7, 673-676.

36. Croop, J. M., et al., (1997) Gene 185, 77-85.

37. Tabas, I. & Tall, A. R. (1984) J. Biol. Chem. 259, 13897-13905.

38. Yancey, P. G., et al., (2003) Arterioscler. Thromb. Vasc. Biol. 23, 712-719.

39. Yancey, P. G., et al., (1996) J. Biol. Chem. 271, 16026-16034.

40. Hao, M., et al., (2002) J. Biol. Chem. 277, 609-617.

41. Stampfer, M. J., et al., (1991) N. Engl. J. Med. 325, 373-381.

42. Knoblauch, H., et al., (2004) Hum. Mol. Genet. 13, 993-1004.

43. Nissen, S. E., et al., (2003) J. Am. Med. Assoc. 290, 2292-2300.

44. Blum, C. B., et al., (1977) J. Clin. Invest. 60, 795-807.

45. Whitlock, M. E., et al., (1989) J. Clin. Invest. 84, 129-137.

46. Brousseau, M. E., et al., (2004) N. Engl. J. Med. 350, 1505-1515.

1. A non-macrophage cell having therein an expression vector encoding ABCG1, wherein the non-macrophage cell expresses ABCG1.

2. The cell of claim 1, wherein the ABCG1 is human ABCG1.

3. The cell of claim 1, wherein the cell is a bacterial cell, a yeast cell, an insect cell or a mammalian cell.

4. The cell of claim 1, wherein the cell is a macrophage-like cell.

5. The cell of claim 1, wherein prior to having the expression vector introduced into it, the non-macrophage cell does not express ABCG1.

6. The cell of claim 1, wherein prior to having the expression vector introduced into it, the non-macrophage cell expresses little ABCG1.

7. The cell of claim 1, wherein prior to having the expression vector introduced into it, the non-macrophage cell does not express any cholesterol efflux-mediating protein.

8. A non-macrophage cell having therein an expression vector encoding ABCG4, wherein the non-macrophage cell expresses ABCG4.

9. The cell of claim 8, wherein the ABCG4 is human ABCG4.

10. The cell of claim 8, wherein the cell is a bacterial cell, a yeast cell, an insect cell or a mammalian cell.

11. The cell of claim 8, wherein the cell is a macrophage-like cell.

12. The cell of claim 8, wherein prior to having the expression vector introduced into it, the non-macrophage cell does not express ABCG4.

13. The cell of claim 8, wherein prior to having the expression vector introduced into it, the non-macrophage cell expresses little ABCG4.

14. The cell of claim 8, wherein prior to having the expression vector introduced into it, the non-macrophage cell does not express any other cholesterol efflux-mediating protein.

15-72. (canceled)
